SEQUENCE LISTING (12 sequences)

SEQ ID NO: 1 - DNA, 4015 bp, Homo sapiens
gcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 2 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 3 - DNA, 20 bp, Artificial Sequence
  primer
ggccaggaattgacgaagtc

SEQ ID NO: 4 - DNA, 26 bp, Artificial Sequence
  primer
acagaatgttgtagagttcaatgcga

SEQ ID NO: 5 - DNA, 19 bp, Artificial Sequence
  primer
gcgattccttcactgatac

SEQ ID NO: 6 - DNA, 20 bp, Artificial Sequence
  primer
gcattatgagacatccccac

SEQ ID NO: 7 - DNA, 19 bp, Artificial Sequence
  primer
gacacgctgcggggcatct

SEQ ID NO: 8 - DNA, 21 bp, Artificial Sequence
  primer
ctgctccccttccttctctca

SEQ ID NO: 9 - DNA, 19 bp, Artificial Sequence
  primer
cacctcacccacgcgaaaa

SEQ ID NO: 10 - DNA, 23 bp, Artificial Sequence
  primer
ccaaagagtttgcgacgcatgtt

SEQ ID NO: 11 - DNA, 21 bp, Artificial Sequence
  primer
cttgctcgagatgtcatgaag

SEQ ID NO: 12 - DNA, 21 bp, Artificial Sequence
  primer
gtttgcattgttttaccagtg

(19) United States
(12) Patent Application Publication
(10) Pub. No.: US 2005/0008621 A1
(43) Pub. Date: Jan. 13, 2005

(54) Preadipocyte cell strains and uses therefore

(76) Inventors: James Kirkland, Brookline, MA (US); Tamara Tchkonia, Brookline, MA (US)

Correspondence Address: LAHIVE & COCKFIELD, LLP., 28 STATE STREET, BOSTON, MA, 02109, US

(21) Appl. No.: 10/492,034

(22) PCT Filed: Oct. 7, 2002
(86) PCT No.: PCT/US02/31635

Related U.S. Application Data
(60) Provisional application No. 60/327,650, filed on Oct. 6, 2001.
(60) Provisional application No. 60/327,651, filed on Oct. 6, 2001.

(51) Int. Cl. A61K 48/00; C12N 5/08

(52) U.S. Cl. 424/93.21; 435/366

(57) ABSTRACT

The present invention relates to preadipocyte strains that maintain replicative potential and adipogenic capacity. In particular, the invention relates to preadipocytes engineered to express telomerase reverse transcriptase (TERT). Use of the cells as research tools, in screening assays, and as therapeutic and/or clinical reagents is also described.

### RELATED APPLICATIONS

This application claims the benefit of and priority to copending provisional patent application U.S. Ser. No. 60/327,650 and U.S. Ser. No. 60/327,651, both filed Oct. 6, 2001. The entire disclosures of the above-referenced applications are incorporated herein by this reference.

### GOVERNMENT RIGHTS

This invention was made with Government Support under Contract Number AG/DK 13925 awarded by the National Institutes of Health. The Government has certain rights in the invention.

### BACKGROUND OF THE INVENTION

Models of adipocyte growth and differentiation have become the focus of intense research in recent years. Not only is the adipocyte vitally important to energy homeostasis, adipose tissue is also believed to play a central role in many of the pathologies associated with obesity and its related disorders. Obesity is one of the most significant health problems in the United States today. About 60 million Americans are overweight and about 35 million of the adult population are obese. Obesity is due, at least in part to an overabundance of fat cells. Obesity, moreover, is considered a major risk factor for noninsulin-dependent diabetes mellitus (NIDDM) and hypertension and has also been linked to various types of cancers as well as immune dysfunction (Moller and Flier (1991) N. Engl. J. Med. 325:938-948; and Spiegelman et al. (1993) J. Biol. Chem. 268:6823-6826).

Consequently, all aspects of adipocyte biology, including adipogenesis, have recently become the targets of intense scientific investigation. Models of adipocyte growth and differentiation are useful in studying the molecular mechanisms of adipocyte development and can lead to a better understanding of the various human disease states in which adipocytes are proposed to play a role. The various models used in studying adipocyte development, include both in vivo studies as well as in vitro cell culture systems.

In vivo studies of adipocyte development have been attempted, but such studies are plagued with a host of difficulties. For example, adipocytes make up only approximately a third of the cells in fat tissue with the remaining two thirds comprising small blood vessels, nerve tissue, fibroblasts and preadipocytes in various stages of development (Géloen et al. (1989) Am. J. Physiol. 257:E547-E553). The distinction between preadipocytes and fibroblasts is difficult to make, and the inability to synchronize preadipocytes at set developmental stages confounds detailed in vivo studies.

Studies using in vitro adipocyte cells lines have produced much of the knowledge relating to adipocyte growth and differentiation. For example, pluripotent fibroblasts (10T1/2, Balb/c 3T3, 1246, RCJ3.1 and CHEF/18 fibroblasts) can be converted upon treatment with 5-azacytidine into several cell types including preadipose, premuscle and precartilage tissue (Taylor and Jones (1979) Cell 17:771-779). Preadipose generated, for example, by 5-azacytidine treatment can be used in further studies of adipogenesis. Moreover, unipotent preadipocytes (3T3-L1, 3T3-F422A, 1246, Ob1771, TA1 and 30A5) that are pre-determined to develop along the adipogenic lineage can be studied as preadipocytes or can be converted to adipose cells using a combination of hormonal inducers.

There are advantages and disadvantages to using cell lines to study adipocyte differentiation. In one respect, cells lines are clonal in nature, allowing one to culture a homogenous population of cells, control the stage of differentiation, and treat the cells with knowledge of the stage of differentiation and of the expected phenotypic responses. Cell lines can also be passaged indefinitely, providing a consistent source of cells for study. Disadvantages associated with using immortal cell lines include reversion to a fibroblastic phenotype at high passage number and potentially inaccurate representation of the true adipogenic phenotype due to the fact that these cells have been rendered immortal.

Recent studies have been aimed at culturing primary preadipocytes. However, the use of primary culture has serious disadvantages. First, it is difficult to isolate preadipocytes from other fibroblast-like cells. Second, large amounts of fat tissue are required because preadipocytes constitute only a small percentage of total fat tissue. In addition, primary cultures have a limited life span and lose adipogenic potential in culture. For detailed reviews of the molecular mechanisms of adipogenesis and these model systems, see Morrison and Farmer (2000) Nutr. 130:3116S-3121S and Natambi and Kim (2000) J Nutr. 130:2122S-3126S, respectively.

Further problems are associated with studies of human primary cells. In particular, human preadipocytes are technically difficult to isolate and culture and only very few laboratories are capable of routinely culturing these cells. As with other human cell types, the capacity of human preadipocytes to replicate declines gradually as they are passaged, but their capacity to differentiate into fat cells declines rapidly. Since it is therefore necessary to use primary or early passage cultures to study adipogenesis, these cells are very expensive as initial collection and isolation account for most of the cost. Culture purity can also be an issue since cultures cannot be derived from single cells and still maintain capacity to differentiate. This, coupled with limited replicative potential, makes it impractical to manipulate and study adipogenic function and mechanisms using, for example, stable transfection approaches. Furthermore, fat specimens are difficult to obtain in quantity, particularly from lean subjects, very old subjects, and fat depots that are difficult to access.

Given the significant drawbacks associated with the model systems described above, there exists a need to develop novel culture systems that accurately mimic in vivo adipocyte development and function, yet are conducive to large-scale and reproducible study.

### SUMMARY OF THE INVENTION

The present invention features preadipocyte cell strains (e.g., primary preadipocyte strains) that maintain replicative potential and adipogenic capacity over many population doublings. In particular, the present inventors have developed a method of generating primary adipocyte cells strains that have been engineered to express the catalytic subunit of telomerase, telomerase reverse transcriptase (TERT). Telomerase reverse transcriptase (TERT) expression in preadipocytes results in both increased replicative capacity and enhanced differentiation of passaged cells into fat cells. Culturing strains engineered according to the methods of the invention overcomes the problematic loss in differentiative ability that routinely occurs with increasing replication. The methods of the invention are particularly useful for generating pure cultures of human preadipocytes (e.g., from various fat depots or from subjects of different ages) that retain the capacity to differentiate following passaging. Cell strains of the invention are particularly useful for studying the effects of fat depot origin and donor age on human fat cell function. Other uses are readily apparent from the instant description including, but not limited to use in identification of agents and/or development of drugs to treat obesity, diabetes and other conditions. Cell strains can be featured in assays to identify agents that function in a fat depot-specific manner. Cell strains can be prepared from subjects of different backgrounds (e.g., different disease states, genetic backgrounds, ages, gender, etc.) to determine the effect of these backgrounds on adipogenesis. Cell strains can also be used in a various clinical and/or therapeutic applications, as described herein. For example, cell strains of the present invention can be used in certain cosmetic applications and/or as biological vehicles for the administration or delivery of therapeutic products (e.g., for the systemic delivery of therapeutic proteins).

At least thirty one telomerase transfected cell strains have been prepared. Cultures that have undergone up to 50 divisions retain capacity to differentiate (e.g., accumulate lipid) when exposed to inducers of differentiation. By comparison, wild type cultures are not capable of accumulating such substantial amounts of lipid after fewer than 10 divisions.

### BRIEF DESCRIPTION OF THE DRAWINGS

FIG. 1 depicts graphically the increase in time for human preadipocytes to reach confluence as a function of passage. Tc=time to reach confluence.

FIG. 2 is a photomicrograph of primary human preadipocytes cultured for 5, 7, 13, 26, 32 and 38 passages.

FIG. 3 is a graph depicting a decrease in glycerol-3-phosphate activity in human preadipocytes with increasing passage number.

FIG. 4 is a photograph depicting decrease in PPARγ2 and C/EBPα mRNA expression with increasing passage of primary preadipocytes. 18S rRNA and HPRT were used as internal controls.

FIG. 5 is a photograph depicting telomere shortening of preadipocytes passaged 32 times.

FIG. 6 is a schematic representation of a preferred plasmid for expressing telomerase reverse transcriptase (TERT) in preadipocytes.

FIG. 7 is a photograph of a blot depicting telomerase reverse transcriptase (TERT) mRNA. hTERT-transfected cells were passaged 10 times before telomerase mRNA was analyzed by RT-PCR. Wild type human preadipocytes do not express endogenous telomerase. Positive control 293 cells are included.

FIG. 8 depicts telomerase activity in hTERT transfected human preadipocytes. A TRAP assay was performed showing that hTERT activity increased with the amount of extract from transfected cells assayed. Boiled extract had no activity and a positive control is shown.

FIG. 9 depicts serially passaged human abdominal subcutaneous preadipocytes. Preadipocytes stably expressing TERT retain their capacity to differentiate for at least 39 population doublings. Capacity for differentiation declines rapidly with passage of wild type preadipocytes.

FIG. 10 is a photograph depicting telomere fragment length in hTERT-transfected preadipocytes passaged 35 times.

FIG. 11 is a photograph of a Northern blot comparing PPARγ2 expression of differentiating primary preadipocytes cultured from subjects of various ages.

FIG. 12 is a photograph of a Southern blot comparing telomere length of differentiating primary preadipocytes cultured from subjects of various ages.

FIG. 13 is a photograph of a Northern blot depicting expression of aP2, C/EBPα and PPARγ2, as telomerase-expressing preadipocytes were serially passaged. 18S rRNA and HPRT were used as internal controls.

### DETAILED DESCRIPTION OF THE INVENTION

Preadipocytes are cells in fat depots that can replicate or differentiate into fat cells. Preadipocytes can be cultured from human fat tissue and can be induced to differentiate using enriched medium. The present inventors have demonstrated that the capacity of cultured human preadipocytes to differentiate into fat cells declines with serial passage, limiting the number of cells that can be obtained from human fat tissue biopsies. To overcome this decline in differentiative capacity, primary human preadipocytes were stably transfected with a gene directing expression of the active subunit of telomerase.

Telomerase is an enzyme that restores telomeric DNA at the ends of chromosomes that is lost with each cell division. Telomeres are nucleoprotein structures comprising repeated TTAGGG sequences and associated proteins that cap the ends of chromosomes. Telomeres prevent chromosomal degradation, recombination, and exposure of uncapped DNA ends to the intracellular environment that could otherwise activate DNA damage checkpoints and cellular stress responses (Bailey et al. (2001) Science 293:2462-2465; Blackburn (2000) Nature 408:53-56; Karlseder et al. (2002) Science 295:2446-2449; Karlseder et al. (1999) Science 283:1321-1325; and von Zglinicki (2001) Cancer Lett. 168:111-116). Telomere shortening increases the risk of cell cycle arrest (Allsopp et al. (1995) Exp. Cell Res. 219:130/136). Telomeric DNA is lost during successive cell divisions unless telomerase, a ribonucleoprotein that adds blocks of telomere repeats, or alternative telomere lengthening pathways are active (Bodner et al. (1998) Science 279:349-352). Under most conditions, somatic cells do not express telomerase. Telomeres are shorter in various tissues from old versus young subjects and telomere length predicts remaining replicative capacity of fibroblasts (Allsop et al. (1992) Proc. Natl. Acad. Sci. 89:10114-10118; Hastie et al. (1990) Nature 345:866-868; and Yang et al. (2001) Mech. Ageing Devel. 122: 1685-1694). Hence, the telomere shortening that occurs with both aging and serial subculturing is associated with cellular dysfunction in somatic cells.

The telomerase-transfected cells of the present invention were able to achieve a greater number of doublings more rapidly than non transfected cells. Moreover, telomerase transfection reduced the loss of capacity of these cells to differentiate into fat cells with increasing passage. This permits preparation of larger numbers of preadipocytes that are capable of differentiating into fat cells from smaller amounts of tissue than using wild type non-telomerase transfected cells. This also permits preparation of pure cultures of human preadipocytes, since useful numbers of differentiated fat cells can be derived from a single preadipocyte.

Without wishing to be bound to any particular theory, it is believed that preadipocyte differentiation, expression of G3PD and other lipogenic genes, and maintenance of fat cell function depend on the two critical adipogenic transcription factors, PPARγ2 and C/EBPα. PPARγ2 expression decreases with aging in subcutaneous fat tissue in primates (Hotta et al. (1999) J. Gerontol. 54A:B183-B188). C/EBPα expression shows a similar trend. Therefore, decreasing expression of PPARγ2 and C/EBPα could occur in human preadipocytes with serial passage, resulting in impaired adipogenesis. It is believed that telomere shortening contributes to impaired adipogenesis through decreasing expression of the adipogenic transcription factors, PPARγ2 and C/EBPα, following repeated cell divisions.

Accordingly, in one embodiment, the invention provides a primary preadipocyte strain that maintains replicative potential and/or maintains adipogenic capacity. The term “preadipocyte” refers to a cell existing in or isolated from fat tissue which is capable of replicating yet is committed to the adipogenic phenotype (i.e., is committed to differentiate into an adipocyte or fat cell). The phrase “maintains replicative potential” means that the cells of the strain are capable of being passaged longer than a comparable wild type preadipocyte cell population, i.e., are capable of more population doublings. The phrase “maintains adipogenic capacity” means that the cells of the strain are capable of exhibiting at least one marker of the adipogenic phenotype for a greater number of population doublings as compared to a comparable wild type preadipocyte cell population. Preferably, the strain is a human preadipocyte strain. The term “human preadipocyte” refers to a preadipocyte existing in or isolated from human fat tissue. The strain preferably, expresses telomerase reverse transcriptase (TERT). More preferably, the TERT is human TERT. In one embodiment, the preadipocyte strain maintains replicative potential and adipogenic capacity over at least 40 population doublings. Preferably, the strain maintains replicative potential and adipogenic capacity over at least 50 population doublings.

The invention also provides primary preadipocyte strains having enhanced replicative potential and maintained adipogenic capacity. Preferably, the strain has enhanced adipogenic potential. The phrase “enhanced replicative potential” means that the cells of the strain are capable of doubling at a faster rate than a comparable wild type preadipocyte cell population and, optionally, are also capable of being passaged longer than a comparable wild type preadipocyte cell population. The term “wild type” refers to a non-altered, non-engineered cell or cell population, preferably a non-altered, non-engineered, primary preadipocyte cell or cell population. The phrase “enhanced adipogenic potential” means that the cells of the strain are capable of differentiating faster (i.e., require a shorter time period after treatment with adipogenic inducers to express at least one adipogenic marker) or better (i.e., at least one adipogenic marker is detectable at a higher level). The term “adipogenic marker” includes any marker or read-out of the adipogenic phenotype including but not limited to adipogenic gene and/or protein expression and/or activity (e.g., adipogenic transcription factors, for example, peroxisome proliferator-activated receptor-gamma (PPARγ) and CCAAT/enhancer binding protein-alpha (C/EBPα), adipogenic signal transduction molecules, for example, the signal transducers and activators of transcription (STATs) including STAT1, STAT5A, and STAT5B, adipsin and aP2, adipocyte determination- and differentiation-dependent factor 1 (ADD1), adipocyte complement-related protein (acrp30), lipogenic genes, enzymes (e.g., glycerol-3-phosphate dehydrogenase, and the like), metabolic gene and/or protein expression and/or activity, metabolic functions (e.g., glucose transport), and microscopically visible markers (e.g., oil droplet formation and/or accumulation). Preferably, the strain expresses telomerase reverse transcriptase (TERT). As used herein, the phrase “expresses telomerase reverse transcriptase” means expresses or produces TERT nucleic acid (e.g., mRNA), expresses or produces TERT polypeptide or protein, or exhibits TERT activity (e.g., maintenance of telomere length). Preferably the strain is a human preadipocyte strain. Even more preferably, the strain expresses human telomerase reverse transcriptase (hTERT). In another embodiment, the invention provides telomerase reverse transcriptase (TERT)-transfected primary preadipocytes, preferably human preadipocyte transfected with human telomerase reverse transcriptase (hTERT). In one embodiment, enhanced replicative potential is measured as the ability to achieve at least 40 population doublings, more preferably 50 population doublings, in less than one year. In more preferred embodiments, the enhanced replicative potential is measured as the ability to achieve at least 40 population doublings, more preferably 50 population doublings, in less than 6 months.

Preferably, the strain has enhanced adipogenic potential, wherein the enhanced adipogenic potential is measured by the ability to maintain expression of adipogenic transcription markers for at least 40 passages. Strains having enhanced adipogenic potential preferably maintain expression of TERT for at least 40 passages.

Also provided are methods for producing primary preadipocyte strains which maintain replicative potential and adipogenic capacity as defined herein. In one embodiment, a method involves engineering primary adipocytes to express telomerase reverse transcriptase (TERT) such that adipocytes maintain replicative potential and adipogenic capacity when cultured under appropriate conditions. Also provided are methods for engineering primary adipocytes to maintain replicative potential and adipogenic capacity, that involve introducing into said adipocytes a nucleic acid that encodes telomerase reverse transcriptase (TERT) and selecting for adipocytes that express TERT. In a preferred embodiment, the adipocyte strain maintains replicative potential and adipogenic capacity over at least 40 population doublings, more preferably over at least 50 population doublings.

Also contemplated are methods for producing primary preadipocyte strains having enhanced replicative potential and maintained adipogenic capacity as defined herein. In one embodiment, a method involves engineering primary adipocytes to express telomerase reverse transcriptase (TERT) such that adipocytes have enhanced replicative potential and maintained adipogenic capacity. Also provided are methods for engineering primary adipocytes having enhanced replicative potential and maintained adipogenic capacity, that involve introducing into said adipocytes a nucleic acid that encodes telomerase reverse transcriptase (TERT) and selecting for adipocytes that express TERT. In a preferred embodiment, enhanced replicative potential is measured by the ability to achieve over at least 40 population doublings, more preferably over at least 50 population doublings, in less than one year. Even more preferably, enhanced replicative potential is measured by the ability to achieve over at least 40 population doublings, more preferably over at least 50 population doublings, in less than six months. Preferably, the strain has enhanced adipogenic potential, wherein the enhanced adipogenic potential is measured by the ability to maintain expression of adipogenic transcription markers for at least 40 passages. Strains having enhanced adipogenic potential preferably maintain expression of TERT for at least 40 passages, and more preferably for at least 50 passages.

In another aspect of the present invention, a preadipocyte strain is obtained by a method that includes introducing into an isolated primary preadipocyte population a nucleic acid that encodes telomerase reverse transcriptase (TERT), selecting from said population a preadipocyte that expresses TERT, and replicating said preadipocyte that expresses TERT. Preferably, the primary adipocytes are human primary adipocytes, and also preferable is that the TERT is human TERT (hTERT).

Also provided are methods to identify adipogenic modulators featuring the strains produced according to the instant methodologies. The methods generally include contacting a strain or a cell from a strain with a test compound, and determining an effect on the cell or strain, such that a modulator is identified. The cell or strain can be used in cosmetic or reconstructive surgery and/or for use in administering a therapeutic agent. In a preferred embodiment, the therapeutic agent is a secreted protein. The protein secretion can be enhanced as compared to a suitable control. For example, if the protein secretion is absent or deficient in a patient, the control can be that patient's own cells, and if it is desired to increase protein secretion as compared to normal secretion, the control can be physiologically representative wild type cells. Various alternative suitable controls can be devised by the skilled practitioner. Therapeutic agents can include, but are not limited to, hormones, growth factors, cytokines, enzymes, cholesterol binding proteins, cholesterol removing proteins, and combinations of these agents. The therapeutic agent can be an adipocytokine, and more preferably, adiponectin. The therapeutic agent also can be insulin. Adiponectin (adipocyte compliment related protein-30 or acrp30) is an adipocytokine that effects obesity related risk factors and is a potential drug candidate for use in treatment of, e.g., diabetes and obesity. Predipocytes can be genetically engineered to express adiponectin by utilizing its known cDNA insert. (Maeda et al. (1996) Biochem. Biophys. Res. Commun. 221(2): 286-289).

Primary preadipocytes that have been co-transfected with telomerase reverse transcriptase (TERT) and a therapeutic polypeptide-encoding nucleic acid are also featured, as are methods for administering the therapeutic polypeptide by administering the co-transfected primary preadipocytes or strains derived therefrom. Preferably, the strain is differentiated prior to administering the strain. Also provided are clinical uses featuring the cells and strains of the invention.

I. Cells to be Engineered

Genetically engineered cells are provided which include a DNA sequence which is expressed by the cell and which codes for a protein having telomerase reverse transscriptase (TERT) activity. The encoded TERT activity is one that is not normally expressed in the cell or that is normally expressed by the cell at only a low level.

In some embodiments, the cells include a genetic (DNA) sequence which is expressed by the cell and which codes for a protein which in the presence of a selected agent results in death of the cell. For example, in some embodiments, the cells include the DNA that encodes a protein that confers upon the cell a resistance to hygromycin or puromycin.

The cells are generally modified in culture using standard in vitro transfection techniques. These modified cells can be used, for example, in screening assays, clinical applications, e.g., cosmetic and reconstructive surgery, and/or for administering therapeutic agents, e.g., therapeutic proteins, to a subject in need thereof. The subject is preferably a human subject but can also be a domesticated animal, for example, a farm animal.

Sources of Cells

The starting populations of preadipocytes can come from a variety of sources. Preferably, the preadipocytes are of human origin, in particular, when used to identify and develop human therapeutic compounds and/or drugs, or when used in clinical applications or to deliver therapeutic proteins in vivo. Preadipocytes of non-human origin can also be used because of the ready availability of such cells in large quantities and at low cost. For example, the cells can be of primate, porcine, bovine or rodent, e.g., murine, origin. Preferably, the preadipocytes are physiologically representative preadipocytes. If human preadipocytes are used as a starting source, e.g., for therapeutic and clinical applications, they are preferably isolated from the subject in need of therapy to prevent immune response against the engineered strains upon administration or transplantation.

The genetically engineered strains for clinical and therapeutic applications are preferably clonally-derived. In this way, the characteristics of the final preparation can be accurately controlled both in terms of the overall properties of the cells and their genetic make-up. Such control is of importance in evaluating the effectiveness of particular treatment protocols and in obtaining regulatory approval for such protocols.

The starting populations of preadipocytes are obtained from fat depots, for example, from abdominal subcutaneous fat, omental and/or mesenteric fat depots (e.g., by surgical removal, biopsy, liposuction, etc.) and are grown and maintained in a tissue culture or other suitable biological medium. Preferably, preadipocytes are isolated from fat tissue biopsies or specimens and cultured. Isolation is achieved by mincing and collagenase digestion. Red blood cells are destroyed using an erythrocyte lysis buffer or other means. Preadipocyte cultures are replated after 6 to 18 hours to permit accurate plating densities and to remove macrophages and mesothelial cells. Preadipocytes are grown to confluence in culture medium, such as alpha minimal essential Eagle's medium containing fetal bovine serum. After subculturing cells for various numbers of passages, cells are transfected as described below. Alternatively, the starting populations of preadipocytes are isolated and frozen in suitable cryogenic media, e.g., for subsequent amplification as required.

It is also within the scope of the invention to generate preadipocyte strains from transgenic non-human mammals having fat cells which overexpress TERT or express heterologous TERT. For example, transgenic non-human mammals having TERT inserted by homologous recombination into chromosomal DNA at the site where the gene is normally located, but under the control of a promoter which enhances expression, or inserted into the chromosome at another locus on the chromosome, are contemplated as sources of starting populations of preadipocytes.

Genetic Engineering

The preadipocytes are genetically engineered so that they express the catalytic or active subunit of telomerase, namely telomerase reverse transcriptase (TERT). Preferably, the cells are genetically engineered to express human telomerase reverse transcriptase (hTERT). DNA sequence information for hTERT has been reported in the literature. The sequence and function of hTERT are described at least, for example, in Nakamura et al. (1997) Science 277:955-959; Meyerson et al. (1997) Cell 90:785-795; Kilian et al. (1997) Hum. Mol. Genet. 6:2011-2019; Wicket al. (1999) Gene 232:97-106; and U.S. Pat. No. 6,261,836, entitled “Telomerase”. The nucleotide and amino acid sequences of hTERT are shown below (SEQ ID NOs:1-2).

TABLE I

Human TERT cDNA Sequence Aquired from
GenBank NM_003219

| | |
|---|---|
| GCAGCGCTGCGTCCTGCTGCGCACGTGGGAAGCCCT | (SEQ ID NO: 1) |
| GGCCCCGGCCACCCCCGCGATGCCGCGCGCTCCCCG | |
| CTGCCGAGCCGTGCGCTCCCTGCTGCGCAGCCACTA | |
| CCGCGAGGTGCTGCCGCTGGCCACGTTCGTGCGGCG | |
| CCTGGGGCCCCAGGGCTGGCGGCTGGTGCAGCGCGG | |
| GGACCCGGCGGCTTTCCGCGCGCTGGTGGCCCAGTG | |
| CCTGGTGTGCGTGCCCTGGGACGCACGGCCGCCCCC | |
| CGCCGCCCCCTCCTTCCGCCAGGTGTCCTGCCTGAA | |
| GGAGCTGGTGGCCCGAGTGCTGCAGAGGCTGTGCGA | |
| GCGCGGCGCGAAGAACGTGCTGGCCTTCGGCTTCGC | |
| GCTGCTGGACGGGGCCCGCGGGGGCCCCCCCGAGGC | |
| CTTCACCACCAGCGTGCGCAGCTACCTGCCCAACAC | |
| GGTGACCGACGCACTGCGGGGGAGCGGGGCGTGGGG | |
| GCTGCTGCTGCGCCGCGTGGGCGACGACGTGCTGGT | |
| TCACCTGCTGGCACGCTGCGCGCTCTTTGTGCTGGT | |
| GGCTCCCAGCTGCGCCTACCAGGTGTGCGGGCCGCC | |
| GCTGTACCAGCTCGGCGCTGCCACTCAGGCCCGGCC | |
| CCCGCCACACGCTAGTGGACCCCGAAGGCGTCTGGG | |
| ATGCGAACGGGCCTGGAACCATAGCGTCAGGGAGGC | |
| CGGGGTCCCCCTGGGCCTGCCAGCCCCGGGTGCGAG | |
| GAGGCGCGGGGGCAGTGCCAGCCGAAGTCTGCCGTT | |
| GCCCAAGAGGCCCAGGCGTGGCGCTGCCCCTGAGCC | |
| GGAGCGGACGCCCGTTGGGCAGGGGTCCTGGGCCCA | |
| CCCGGGCAGGACGCGTGGACCGAGTGACCGTGGTTT | |
| CTGTGTGGTGTCACCTGCCAGACCCGCCGAAGAAGC | |
| CACCTCTTTGGAGGGTGCGCTCTCTGGCACGCGCCA | |
| CTCCCACCCATCCGTGGGCCGCCAGCACCACGCGGG | |
| CCCCCCATCCACATCGCGGCCACCACGTCCCTGGGA | |
| CACGCCTTGTCCCCCGGTGTACGCCGAGACCAAGCA | |
| CTTCCTCTACTCCTCAGGCGACAAGGAGCAGCTGCG | |
| GCCCTCCTTCCTACTCAGCTCTCTGAGGCCCAGCCT | |
| GACTGGCGCTCGGAGGCTCGTGGAGACCATCTTTCT | |
| GGGTTCCAGGCCCTGGATGCCAGGGACTCCCCGCAG | |
| GTTGCCCCGCCTGCCCCAGCGCTACTGGCAAATGCG | |
| GCCCCTGTTTCTGGAGCTGCTTGGGAACCACGCGCA | |
| GTGCCCCTACGGGGTGCTCCTCAAGACGCACTGCCC | |
| GCTGCGAGCTGCGGTCACCCCAGCAGCCGGTGTCTG | |
| TGCCCGGGAGAAGCCCCAGGGCTCTGTGGCGGCCCC | |
| CGAGGAGGAGGACACAGACCCCCGTCGCCTGGTGCA | |
| GCTGCTCCGCCAGCACAGCAGCCCCTGGCAGGTGTA | |
| CGGCTTCGTGCGGGCCTGCCTGCGCCGGCTGGTGCC | |
| CCCAGGCCTCTGGGGCTCCAGGCACAACGAACGCCG | |
| CTTCCTCAGGAACACCAAGAAGTTCATCTCCCTGGG | |
| GAAGCATGCCAAGCTCTCGCTGCAGGAGCTGACGTG | |
| GAAGATGAGCGTGCGGGACTGCGCTTGGCTGCGCAG | |
| GAGCCCAGGGGTTGGCTGTGTTCCGGCCGCAGAGCA | |
| CCGTCTGCGTGAGGAGATCCTGGCCAAGTTCCTGCA | |
| CTGGCTGATGAGTGTGTACGTCGTCGAGCTGCTCAG | |
| GTCTTTCTTTTATGTCACGGAGACCACGTTTCAAAA | |
| GAACAGGCTCTTTTTCTACCGGAAGAGTGTCTGGAG | |
| CAAGTTGCAAAGCATTGGAATCAGACAGCACTTGAA | |
| GAGGGTGCAGCTGCGGGAGCTGTCGGAAGCAGAGGT | |
| CAGGCAGCATCGGGAAGCCAGGCCCGCCCTGCTGAC | |
| GTCCAGACTCCGCTTCATCCCCAAGCCTGACGGGCT | |
| GCGGCCGATTGTGAACATGGACTACGTCGTGGGAGC | |
| CAGAACGTTCCGCAGAGAAAAGAGGGCCGAGCGTCT | |
| CACCTCGAGGGTGAAGGCACTGTTCAGCGTGCTCAA | |
| CTACGAGCGGGCGCGGCGCCCCGGCCTCCTGGGCGC | |
| CTCTGTGCTGGGCCTGGACGATATCCACAGGGCCTG | |
| GCGCACCTTCGTGCTGCGTGTGCGGGCCCAGGACCC | |
| GCCGCCTGAGCTGTACTTTGTCAAGGTGGATGTGAC | |
| GGGCGCGTACGACACCATCCCCCAGGACAGGCTCAC | |
| GGAGGTCATCGCCAGCATCATCAAACCCCAGAACAC | |
| GTACTGCGTGCGTCGGTATGCCGTGGTCCAGAAGGC | |
| CGCCCATGGGCACGTCCGCAAGGCCTTCAAGAGCCA | |
| CGTCTCTACCTTGACAGACCTCCAGCCGTACATGCG | |
| ACAGTTCGTGGCTCACCTGCAGGAGACCAGCCCGCT | |
| GAGGGATGCCGTCGTCATCGAGCAGAGCTCCTCCCT | |
| GAATGAGGCCAGCAGTGGCCTCTTCGACGTCTTCCT | |
| ACGCTTCATGTGCCACCACGCCGTGCGCATCAGGGG | |
| CAAGTCCTACGTCCAGTGCCAGGGGATCCCGCAGGG | |
| CTCCATCCTCTCCACGCTGCTCTGCAGCCTGTGCTA | |
| CGGCGACATGGAGAACAAGCTGTTTGCGGGGATTCG | |
| GCGGGACGGGCTGCTCCTGCGTTTGGTGGATGATTT | |
| CTTGTTGGTGACACCTCACCTCACCCACGCGAAAAC | |
| CTTCCTCAGGACCCTGGTCCGAGGTGTCCCTGAGTA | |
| TGGCTGCGTGGTGAACTTGCGGAAGACAGTGGTGAA | |
| CTTCCCTGTAGAAGACGAGGCCCTGGGTGGCACGGC | |
| TTTTGTTCAGATGCCGGCCCACGGCCTATTCCCCTG | |
| GTGCGGCCTGCTGCTGGATACCCGGACCCTGGAGGT | |
| GCAGAGCGACTACTCCAGCTATGCCCGGACCTCCAT | |
| CAGAGCCAGTCTCACCTTCAACCGCGGCTTCAAGGC | |
| TGGGAGGAACATGCGTCGCAAACTCTTTGGGGTCTT | |
| GCGGCTGAAGTGTCACAGCCTGTTTCTGGATTTGCA | |
| GGTGAACAGCCTCCAGACGGTGTGCACCAACATCTA | |
| CAAGATCCTCCTGCTGCAGGCGTACAGGTTTCACGC | |
| ATGTGTGCTGCAGCTCCCATTTCATCAGCAAGTTTG | |
| GAAGAACCCCACATTTTTCCTGCGCGTCATCTCTGA | |
| CACGGCCTCCCTCTGCTACTCCATCCTGAAAGCCAA | |
| GAACGCAGGGATGTCGCTGGGGGCCAAGGGCGCCGC | |
| CGGCCCTCTGCCCTCCGAGGCCGTGCAGTGGCTGTG | |
| CCACCAAGCATTCCTGCTCAAGCTGACTCGACACCG | |
| TGTCACCTACGTGCCACTCCTGGGGTCACTCAGGAC | |
| AGCCCAGACGCAGCTGAGTCGGAAGCTCCCGGGGAC | |
| GACGCTGACTGCCCTGGAGGCCGCAGCCAACCCGGC | |
| ACTGCCCTCAGACTTCAAGACCATCCTGGACTGATG | |
| GCCACCCGCCCACAGCCAGGCCGAGAGCAGACACCA | |
| GCAGCCCTGTCACGCCGGGCTCTACGTCCCAGGGAG | |
| GGAGGGGCGGCCCACACCCAGGCCCGCACCGCTGGG | |
| AGTCTGAGGCCTGAGTGAGTGTTTGGCCGAGGCCTG | |
| CATGTCCGGCTGAAGGCTGAGTGTCCGGCTGAGGCC | |
| TGAGCGAGTGTCCAGCCAAGGGCTGAGTGTCCAGCA | |
| CACCTGCCGTCTTCACTTCCCCACAGGCTGGCGCTC | |
| GGCTCCACCCCAGGGCCAGCTTTTCCTCACCAGGAG | |
| CCCGGCTTCCACTCCCCACATAGGAATAGTCCATCC | |
| CCAGATTCGCCATTGTTCACCCCTCGCCCTGCCCTC | |
| CTTTGCCTTCCACCCCCACCATCCAGGTGGAGACCC | |
| TGAGAAGGACCCTGGGAGCTCTGGGAATTTGGAGTG | |
| ACCAAAGGTGTGCCCTGTACACAGGCGAGGACCCTG | |
| CACCTGGATGGGGGTCCCTGTGGGTCAAATTGGGGG | |
| GAGGTGCTGTGGGAGTAAAATACTGAATATATGAGT | |
| TTTTCAGTTTTGAAAAAAA | |

TABLE II

Human TERT Amino Acid Sequence Aquired from
GenBank NP_003210

| | |
|---|---|
| MPRAPRCRAVRSLLRSHYREVLPLATFVRRLGPQGW | (SEQ ID NO: 2) |
| RLVQRGDPAAFRALVAQCLVCVPWDARPPPAAPSFR | |
| QVSCLKELVARVLQRLCERGAKNVLAFGFALLDGAR | |
| GGPPEAFTTSVRSYLPNTVTDALRGSGAWGLLLRRV | |
| GDDVLVHLLARCALFVLVAPSCAYQVCGPPLYQLGA | |
| ATQARPPPHASGPRRRLGCERAWNHSVREAGVPLGL | |
| PAPGARRRGGSASRSLPLPKRPRRGAAPEPERTPVG | |
| QGSWAHPGRTRGPSDRGFCVVSPARPAEEATSLEGA | |
| LSGTRHSHPSVGRQHHAGPPSTSRPPRPWDTPCPPV | |
| YAETKHFLYSSGDKEQLRPSFLLSSLRPSLTGARRL | |
| VETIFLGSRPWMPGTPRRLPRLPQRYWQMRPLFLEL | |
| LGNHAQCPYGVLLKTHCPLRAAVTPAAGVCAREKPQ | |
| GSVAAPEEEDTDPRRLVQLLRQHSSPWQVYGFVRAC | |
| LRRLVPPGLWGSRHNERRFLRNTKKFISLGKHAKLS | |
| LQELTWKMSVRDCAWLRRSPGVGCVPAAEHRLREEI | |
| LAKFLHWLMSVYVVELLRSFFYVTETTFQKNRLFFY | |
| RKSVWSKLQSIGIRQHLKRVQLRELSEAEVRQHREA | |
| RPALLTSRLRFIPKPDGLRPIVNMDYVVGARTFRRE | |
| KRAERLTSRVKALFSVLNYERARRPGLLGASVLGLD | |
| DIHRAWRTFVLRVRAQDPPPELYFVKVDVTGAYDTI | |
| PQDRLTEVIASIIKPQNTYCVRRYAVVQKAAHGHVR | |
| KAFKSHVSTLTDLQPYMRQFVAHLQETSPLRDAVVI | |
| EQSSSLNEASSGLFDVFLRFMCHHAVRIRGKSYVQC | |
| QGIPQGSILSTLLCSLCYGDMENKLFAGIRRDGLLL | |
| RLVDDFLLVTPHLTHAKTFLRTLVRGVPEYGCVVNL | |
| RKTVVNFPVEDEALGGTAFVQMPAHGLFPWCGLLLD | |
| TRTLEVQSDYSSYARTSIRASLTFNRGFKAGRNMRR | |
| KLFGVLRLKCHSLFLDLQVNSLQTVCTNIYKILLLQ | |
| AYRFHACVLQLPFHQQVWKNPTFFLRVISDTASLCY | |
| SILKAKNAGMSLGAKGAAGPLPSEAVQWLCHQAFLL | |
| KLTRHRVTYVPLLGSLRTAQTQLSRKLPGTTLTALE | |
| AAANPALPSDFKTILD | |

Reagents for introduction of the sequences encoding TERT in cells are described in the references described above. Preferably, a TERT cDNA is subcloned into a plasmid-based vector which encodes elements for efficient expression in the genetically engineered cell. The plasmid-based vector preferably contains a marker such as a hygromycin or puromycin resistance gene for selection of stable transfectants with the appropriate cytotoxic agents. The plasmid-based vector optionally contains, for example, an ampicillin gene for plasmid selection in bacteria. A preferred reagent for introduction of sequences encoding TERT is a plasmid that contains a TERT cDNA (e.g., a hTERT cDNA) and hygromycin resistance gene.

As known in the art, transfection can be accomplished by electroporation, calcium phosphate precipitation, a lipofectin-based procedure, or microinjection or through use of a “gene gun”. Preferably, a lipid emulsion or calcium phosphate precipitation is used. Transfected cultures are then allowed to divide and are treated with concentrations of, for example, hygromycin or puromycin that are sufficient to kill cells not expressing the hygromycin or puromycin resistance genes.

Expression of telomerase by the remaining cells is tested by measuring telomerase messenger RNA abundance or telomerase activity. Cells expressing this activity are then serially passaged, with earlier passage cells being frozen for future use. Alternatively, the passaged cells are exposed to a differentiation medium that contains insulin, glucocorticoids, glucose, growth factors, and other agents. Cell strains that respond to this medium by accumulating lipid are selected for future use.

Telomerase-expressing nucleic acids can, alternatively, be introduced into preadipocytes by infection. Infection, is accomplished by incorporating the genetic sequence for TERT (e.g., hTERT) into a retroviral vector. Various procedures are known in the art for such incorporation. One such procedure which has been widely used in the art employs a defective murine retrovirus, Psi-2 cells for packaging the retrovirus, and the amphotropic packaging cell line Psi-AM to prepare infectious amphotropic virus for use in infecting the target donor cells, as described by Kohn et al. (1987) Blood Cells 13:285-298. Alternatively, rather than a defective Moloney murine retrovirus, a retrovirus of the self-inactivating and double-copy type can be used, such as that described by Hantzopoulos et al. (1989) Proc. Natl. Acad. Sci. USA 86:3519-3523.

A variety of methods are known to those skilled in the art for making transgenic animals expressing TERT as a source of cells from which to derive modified cell strains. Examples of particularly useful animals include rabbits and pigs, although transgenic mice, rats, rabbits, pigs, sheep, and cattle have been made using standard techniques. The most well known method for making a transgenic animal is by superovulation of a donor female, surgical removal of the egg and injection of the genetic material in the pronuclei of the embryo, as taught by U.S. Pat. No. 4,873,191 to Wagner, the teachings of which are incorporated herein. Another commonly used technique involves the genetic manipulation of embryonic stem cells (ES cells). Briefly, ES cells are grown and maintained in a plueripotent state. Genetic material is introduced into the embryonic stem cells, for example, by electroporation according to the method of McMahon and Bradley (1991) Cell 62:1073-1085. Colonies are picked from day 6 to day 9 of selection and expanded and used to isolate DNA for Southern blot analysis.

Culturing and Storage of Developed Preadipocyte Strains

After being genetically engineered in the manner described above, the resulting strains are normally stored in liquid nitrogen tanks until needed for use as research tools, in screening assays, in clinical application, (e.g., cosmetic and/or reconstructive surgery), or for treatment of a particular subject (e.g., therapeutic treatment). Preferably, early passage cultures of preadipocyte strains are frozen for subsequent amplification as required. Cells are preferably maintained in the undifferentiated phenotype during development, avoiding extensive cell-cell contact, confluence, and growth factor depletion of culture media. The ability to prepare preadipocyte strains in advance and store them until needed is an important advantage.

It is not anticipated to be possible to propagate the preadipocyte strains indefinitely (since they are not transformed). It is anticipated that each 25 cm² flask prepared from a single originating cell will ultimately be able to undergo at least 10 passages at a 1:2 split ratio and maintain ability to differentiate, yielding over 1000 25 cm² flasks per transfectant. Strains produced according to the present methodologies, are anticipated to produce sufficient cells to conduct research studies, pharmacological screening and/or clinical and therapeutic approaches, as described herein.

Preferred Features of Telomerase-Transfected Strains

Telomerase transfected serially passaged human preadipocytes have an increased capacity to differentiate into fat cells compared to wild type cells. Primary cultures derived from fat tissue can be contaminated with cell types other than preadipocytes. By using telomerase transfected cells that retain capacity to differentiate into fat cells after many divisions, preparation of larger numbers of cultured fat cells from purer cultures of preadipocytes (since cultures can be grown from single cells) can be achieved. This facilitates use of these cells in, for example, drug discovery or determination of human adipocyte-specific gene products or released proteins.

Preadipocytes from different fat depots can be transfected permitting discovery of fat depot specific agents or genes expressed or proteins released in a depot specific manner. Telomerase transfected preadipocytes can be prepared from subjects of different genetic backgrounds, ages, gender, and degrees of obesity and subjects with different diseases, such as diabetes, to determine effects of these on, for example, fat cell response to agents, gene expression, or released proteins.

Moreover, further aspects of the present invention include: preparation of preadipocyte cultures from single cells that retain the ability to differentiate into fat cells, yielding pure cultures (primary cultures derived from fat tissue can be contaminated with cell types other than preadipocytes); use of these pure preadipocyte cultures in drug discovery, by applying agents to the cells and determining effect on fat accumulation, release of proteins, or other cell functions; use of these cultures to discover molecules, such as peptides of proteins, released by fat cells; use of these cultures to determine fat depot specific functions, such as responses to drugs (for example, drugs to treat fat accumulation in specific fat depots), genes expressed, or molecules released; and use of passaged cells from a subject that are differentiated and then transplanted into the subject for cosmetic or restorative surgery.

II. Research, Screening, Clinical and Therapeutic Applications

As discussed above, the engineered cells can be used in various research applications, in screening assays (e.g., for the identification and development of reagents for modulating adipogenesis) and in clinical applications, for example, in cosmetic surgery and/or for the administration of therapeutic proteins.

Research Applications

The genetically engineered strains of the invention are useful as research tools for investigators seeking to more fully understand the mechanisms of adipocyte growth, differentiation, development and function, as well as to identify biological properties and adipogenic processes which may contribute to various disorders or disease states associated with adipogenic cells. In one embodiment, preadipocyte strains are isolated from age-disparate subjects. In another embodiment, preadipocyte strains are isolated from regions of varying adipogenic function, (e.g., from abdominal subcutaneous, omental, and mesenteric fat depots). Sufficient cells are produced to conduct pharmacological and transient transfection studies of metabolic and other pathways, and as a model system for determining mechanisms of regional and aging differences in fat tissue function under highly controlled conditions.

Genetically engineered strains representing regional and/or aging differences in fat tissue function are also extremely useful for the identification of druggable targets, e.g., targets for use in the screening assays described below.

Screening Assays

The genetically engineered strains of the invention are also extremely useful in methods (also referred to herein as a “screening assays”) for identifying compounds and/or agents, ie., candidate or test compounds or agents (e.g., peptides, peptidomimetics, small molecules or other drugs) which modulate adipogenesis. As used herein, a compound which modulates adipogenesis is any compound or agent effective at altering (e.g., increasing, enhancing, upregulating, decreasing, inhibiting, downregulating, etc.) at least one activity, function, pathway or mechanism associated with, or characteristic of, adipocytes. In one embodiment, the invention provides assays for screening candidate or test compounds which modulate at least one activity, function, pathway or mechanism selected from the group consisting of (1) lipid metabolism; (2) storage of free fatty acid (FFA) as triacylglycerol (TAG); (3) glucose metabolism and/or transport (e.g., expression and/or activity of the insulin-dependent glucose transporter, Glut4); (4) endocrine functions including but not limited to secretion of angiotensinogen (AGT), plasminogen activator inhibitor type 1 (PAI-1), tumor necrosis factor α (TNFα), interleukin-6 (IL-6), adipsin and adipocyte complement-related protein (acrp30); (5) lipid synthesis and/or lipid accumulation); (6) fatty acid synthesis; (7) fat-specific gene and/or protein expression; (8) hormone- (e.g., insulin-) mediated signaling (e.g., Janus kinase-2/signal transduction and activators of transcription (JAK-2/STAT) signaling, mitogen-activated protein kinase (MAPK) signaling and phosphoinositide 3-kinase (PI3 kinase)/p70 S6 kinase signaling); (9) energy storage; (10) secretion (e.g., secretion of paracrine and/or endocrine factors); (11) adipocyte growth; (12) adipocyte differentiation; and (13) adipogenic development.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145). Examples of methods for the synthesis of molecular libraries can be found in the art, for example in DeWitt et al. (1993) Proc. Natl. Acad. Sci. U.S.A. 90:6909; Erb et al. (1994) Proc. Natl. Acad. Sci. USA 91:11422; Zuckermann et al.(1994). J. Med. Chem. 37:2678; Cho et al. (1993) Science 261:1303; Carrell et al.(1994) Angew. Chem. Int. Ed. Engl. 33:2059; Carell et al. (1994) Angew. Chem. Int. Ed. Engl. 33:2061; and Gallop et al. (1994) J. Med. Chem. 37:1233. Libraries of compounds may be presented in solution (e.g., Houghten (1992) Biotechniques 13:412-421), or on beads (Lam (1991) Nature 354:82-84), chips (Fodor (1993) Nature 364:555-556), bacteria or spores (Ladner U.S. Pat. No. 5,223,409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra).

In one embodiment, an assay is a cell-based assay in which a cell (e.g., a cell population or strain of the invention), is contacted with a test compound and the ability of the test compound to modulate an adipogenic activity is determined. Determining the ability of the test compound to modulate an adipogenic activity can be accomplished by according to any art-recognized activity assay methodology. Assays for agonists and antagonists of any of the adipogenic activities are contemplated within the scope of this invention. Candidate compounds can be identified as adipogenic modulators based on comparison of the adipogenic activity in the presence of the test compound as compared to an appropriate control. The term “appropriate control”, as defined herein, includes any control recognized by the skilled artisan to assist in the identification of a test compound as a candidate modulatory compound. Appropriate controls include, but are not limited to, wells, cells or samples treated with buffers or solvents (e.g., buffers or solvents used to suspend or dissolve the test compounds being screened), media or other physiologically-tolerable reagents, blanks, predetermined values (e.g., predetermined positive and/or negative or graded values), and the like. Comparison can also be made between test compounds.

The invention further pertains to novel agents identified by the above-described screening assays. It is also within the scope of this invention to further use an agent identified as described herein in an appropriate animal model. For example, an agent identified as described herein can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

Therapeutic and Clinical Applications

The genetically engineered strains of the invention also provide an excellent mechanism for the administration of therapeutic agents either locally at the site of cell implantation or systemically. The strains of the invention can be engineered, for example, to secrete a desired therapeutic protein. Protein delivery via engineered strains offer several advantages including direct secretion of therapeutic protein products without any barrier to diffusion. Adipogenic cells and/or strains are also particularly suited for use as therapeutic protein delivery vehicles due to their excellent secretory properties. Examples of the types of therapeutic agents which can be administered in this way include hormones, growth factors, cytokines, enzymes, cholesterol binding or removing proteins, and the like. A particularly preferred therapeutic agent is insulin. In each case, the gene encoding the desired therapeutic protein is introduced into the strain expressing TERT (or co-introduced into a starting population of preadipocytes), prior to transplantation. Preferably, strains co-expressing TERT and the desired therapeutic agent are differentiated prior to transplantation.

Moreover, the genetically engineered cells described herein provide an important solution for addressing problems associated with reconstructive surgery. For example, cells can be isolated from the subject in need of reconstructive surgery and adequate supplies can be generated before the surgical procedure.

III. Methods

The following are exemplary, non-limiting methods of preadipocyte culture, differentiation, transfection, and cloning in accordance with the present invention, as well as exemplary methods of analyses of G3PD, DNA, RNA, telomere restriction fragmentation, and telomerase activity.

Human Subjects

Fat tissue was resected during gastric bypass surgery for management of obesity from 14 subjects who had given informed consent. The protocol was approved by the Boston University Medical Center Institutional Review Board for Human Research. All subjects had fasted at least 10 hours. Thirteen of the subjects were women. Subjects were 42±5 years of age (mean±1 SEM; range 18-69). The subjects' mean body mass index (BMI) was 50±5 kg/m². Subjects with malignancies were excluded. No subjects were taking thiazolidinediones or steroids. None had fasting plasma glucose levels over 120 mg %. Two to 10 g of abdominal subcutaneous (external to the fascia superficialis) fat was obtained from each subject.

Preadipocyte Culture

Fat tissue was minced and digested in Hank's balanced salt solution containing 1 mg/ml collagenase and 7.5% fetal bovine serum (FBS) in a 37° C. shaking water bath until fragments were no longer visible and the digest had a milky appearance. Digests were filtered and centrifuged at 800×G for 10 min. The digests were treated with an erythrocyte lysis buffer to improve subsequent differentiation (Hauner et al. (1989) J. Clin. Invest. 84:1663-1670; and Van de Ventner et al. (1994) J. Cell. Biochem. 54:1-10). The cells were then plated using a low serum plating medium (1:1 Dulbecco's modified Eagle's medium (DMEM): Ham's F12 that contained 0.5% bovine serum and antibiotics) at a density of 4×10⁴ cells/cm². Macrophages were rare (less than 5 per 10⁶ cells as assessed by phase contrast microscopy) in the replated cultures. Plating medium was changed every 2 days until confluence. Cultures were serially passaged by replating confluent cells at half their confluent density, growing the cells to confluence, and replating again at half their confluent density.

Preadipocyte Differentiation

From confluence, cells were either held in an undifferentiated state using plating medium without serum, serially-passaged, or differentiated. For differentiation, a previously published method (Hauner et al. (1995) Diabetelogia 38: 764-771) was used with modifications that included the following. Cultures were treated for 7 to 14 days with plating medium (without serum) enriched with 100 nM dexamethasone, 500 nM human insulin, 200 pM triiodothyronine, 0.5 μM rosiglitazone, antibiotics, and 540 μM methylisobutylxanthine (removed after 2 days). In preliminary studies, higher rosiglitazone and insulin concentrations did not further enhance differentiation. Medium was changed every 2 days.

Preadipocyte Transfection

Preadipocytes were isolated from a 44 year old female subject. After cells had undergone 7 population doublings, they were transfected with the plasmid, pBABE-hTERT-Hygro (Counter et al. (1998) Oncongene 16:1217-1222). This plasmid expresses the human telomerase catalytic component driven by the Moloney murine leukemia virus long terminal repeat promoter and a hygromycin resistance sequence driven by the SV40 promoter. 22 stably transfected, hygromycin-resistant clones were selected.

Preadipocyte Cloning

Wild type or telomerase-transfected preadipocytes cultured as described above were replated at a density of 50 cells/96 well plate in plating medium. At this density, the probability of any one well's being seeded by more than one cell is less than 2% (Kirkland et al. (1990) Am. J. Physiol 258:C206-C210; and Wang et al. (1989) J. Clint. Invest. 83:1741-1746). After 2 weeks, colonies were evident and by 3 weeks, some of the telomerase-expressing clones were confluent.

Glycerol-3-Phosphate Dehydrogenase (G3PD) and DNA Assays

G3PD was measured in supernatants of cell homogenates by following NADH disappearance spectrophotometrically (Kozak et al. (1974) J. Biol. Chem. 249:7775-7781; and Kirkland et al. (1987) J. Cell. Physiol. 133:449-460). G3PD activity was not detectable in undifferentiated preadipocytes. DNA was measured in homogenates using a fluorimetric intercalating dye reaction (Labarca et al. (1980) Anal. Biochem. 102:344-352). Cell numbers in confluent cultures estimated by this method agreed within 3% of directly counted cell numbers.

RNA Analysis

For RNA analyses, RNA was isolated from preadipocytes using the guanidinium thiocyanate-phenol method (Chomczynski et al. (1987) Anal. Biochem. 162:156-159). RNA integrity was verified using 1% formaldehyde-containing denaturing agarose gels. Messenger RNA (mRNA) was measured by relative quantitative RT-PCR in which target genes were coamplified with internal control sequences (18S rRNA or hypoxanthine phosphoribosyl transferase [HPRT])(Morin et al. (1997) J. Gerontol. 52A:B 190-B195). Analysis of mRNA expression was carried out during the exponential phase of the amplification, which was assessed in preliminary experiments for each set of primers. Amplified product reproducibility was confirmed by two PCR rounds. The ratios of intensity of target to internal control bands were used to indicate the relative abundance of message in the samples. This allows quantitative data to be obtained since 18S rRNA and HPRT abundance are not affected by passage or differentiation of preadipocytes. 18S rRNA amplification was titrated to match that of adipocyte fatty acid binding protein (aP2; a differentiation-dependent target of PPARγ2 and C/EBPα) and PPARγ2 mRNA by adding competitive primers (Ambion, Austin, Tex.) that modulate extension of the 18S cDNA. HPRT abundance was close to that of C/EBPα. The quantitative nature of this approach was confirmed by measuring the aP2, PPARγ2, and C/EBPα mRNA's in serially diluted samples. RNA preparations were checked for DNA contamination by amplifying control aliquots that had not been reverse-transcribed. The following primers were used: for aP2, sense, GGCCAGGAATTGACGAAGTC (SEQ ID NO: 3), and antisense, ACAGAATGTTGTAGAGTTCAATGCGA (SEQ ID NO: 4) (Sen et al. (2001) J. Cell. Biochem. 81:312-319); for PPARγ2, sense, GCGATTCCTTCACTGATAC (SEQ ID NO: 5), and antisense, GCATTATGAGACATCCCCAC (SEQ ID NO: 6) (Auboeuf et al. (1997) Diabetes 46:1319-1327); for C/EBPα, sense, GACACGCTGCGGGGCATCT (SEQ ID NO: 7), and antisense, CTGCTCCCCTTCCTTCTCTCA (SEQ ID NO: 8) (Zilberfarb et al. (2001) Diabetologia 44:377-386); for hTERT, sense CACCTCACCCACGCGAAAA (SEQ ID NO: 9), and antisense, CCAAAGAGTTTGCGACGCATGTT (SEQ ID NO: 10) (Yang, J. et al. (1999) J. Biol. Chem. 274:26141-26148); and for HPRT sense, CTTGCTCGAGATGTCATGAAG (SEQ ID NO: 11), and antisense GTTTGCATTGTTITACCAGTG (SEQ ID NO: 12) (based on sequence accession No. J00423).

Telomere Restriction Fragment Length Assay

Telomere length was measured using a terminal restriction fragment length (TRF) assay (Harley et al. (1990) Nature 345:458:460) using a kit (Roche, Mannheim, Germany). Briefly, genomic DNA was isolated (Qiagen DNA isolation kit, Valencia, Calif.). DNA was digested using a Rsa1/Hinf1 mixture that cuts extratelomeric DNA frequently, but does not have target sequences in telomeric and subtelomeric DNA. DNA fragments were resolved on a 0.7% agarose gel and transferred to a membrane. Membranes were hybridized using a digoxigenin-labeled probe specific for telomeric repeats. The hybridized probe was detected by adding alkaline phosphatase to generate a chemiluminescent product. Following densitometry, mean TRF length was calculated by comparing signal intensity to molecular weight standards (Oullette et al. (2000) J. Biol. Chem. 275:10072-10076).

Terminal Repeat Amplification Protocol (TRAP) Assay

Telomerase activity was assayed using a PCR-based TRAP protocol (Kim et al. (1994) Science 266:2011-2015) using a kit (TRAPeze, Intergen, Purchase, N.Y.). Briefly, telomerase activity in cell extracts was determined through its ability to synthesize telomeric repeats onto an oligonucleotide substrate in vitro. The product was then amplified by PCR. The PCR product was run on a 15% nondenaturing acrylamide gel and visualized by SYBR Green staining. In each assay, extracts equivalent to 2,000-10,000 cells were used.

Statistical Analysis

Results are means±1 SEM and significance determination was by paired T tests (Kachigan et al. (1986) Statistical Analysis New York:Radius Press; and Keppel et al. (1973) A Researcher's handbook New Jersey:Prentice-Hall, Inc.). Two-tailed P<0.05 was considered significant.

The present invention will be more fully described by the following non-limiting examples.

### EXAMPLES

### Example I

### Restricted Replicative Potential and Declining Capacity of Primary Adipocytes

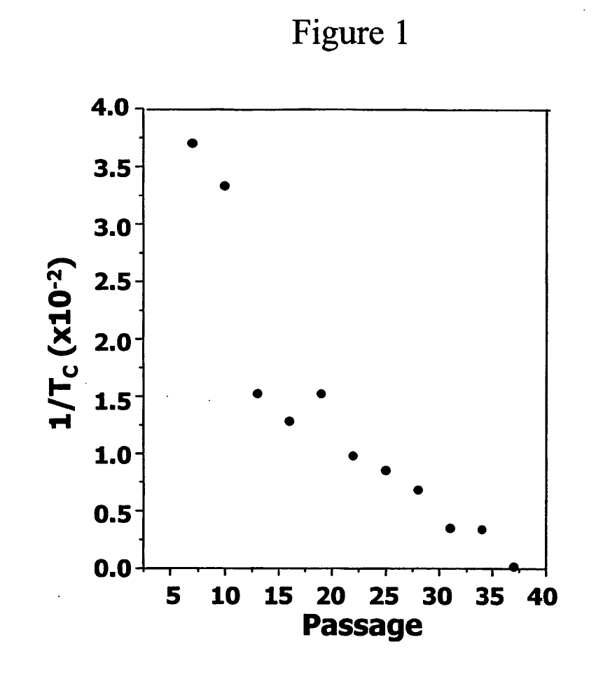

Human preadipocytes are technically difficult to isolate and culture. Moreover, as with other human cell types, the capacity of human preadipocytes to replicate (i.e., their replicative capacity) declines gradually as they are passaged. Primary, wild type abdominal subcutaneous preadipocytes were passaged at a 1:2 split ratio. The time to confluence (T_{c}) increased as a function of passage until cells were no longer able to achieve confluence by passage 36±3. This took over 2 years. These data are set forth in FIG. 1. The time required for primary preadipocytes to undergo the first 5 passages was 38±5 days while the time taken to progress from passage 6 to 10 was 75±14 days (N=11 different fat samples; P<0.01; paired T test). Cells were no longer capable of reaching confluence after 36±3 passages (N=3 experiments), which took over 2 years to achieve. Together with declining replicative potential with serial passage, capacity for differentiation into fat cells declined. Human omental preadipocytes took longer than subcutaneous preadipocytes to achieve 10 population doublings.

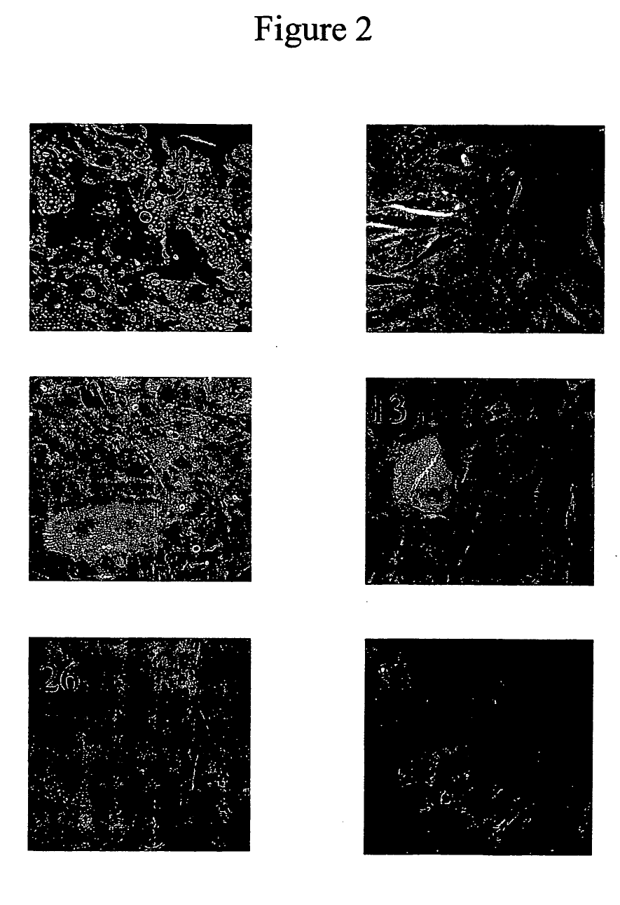

More problematic, however, is the fact that the capacity of human preadipocytes to differentiate into fat cells declines rapidly as cells are passaged. FIGS. 2-4 demonstrate that as preadipocytes were serially passaged, increases in lipid accumulation, glycerol-3-phosphate dehydrogenase activity, C/EBPα and PPARγ expression following exposure to differentiation inducers declined.

First, 5th, 7th, 13th, 26th, 32nd and 38th passage wild type abdominal subcutaneous preadipocytes were exposed to differentiation-inducing medium for 10 days. As can be seen by the data in FIG. 2, cells that had undergone 7 population doublings before treatment with differentiation-inducing medium exhibited more morphologically evident adipogenesis than cells that had undergone 13 doublings. By the time they had achieved 26 and 32 population doublings, preadipocytes from the same subject could only accumulate very little lipid, and cells no longer produced fat droplets upon hormone induction. With increasing passage, the cells became flattened with large nuclei and spindling.

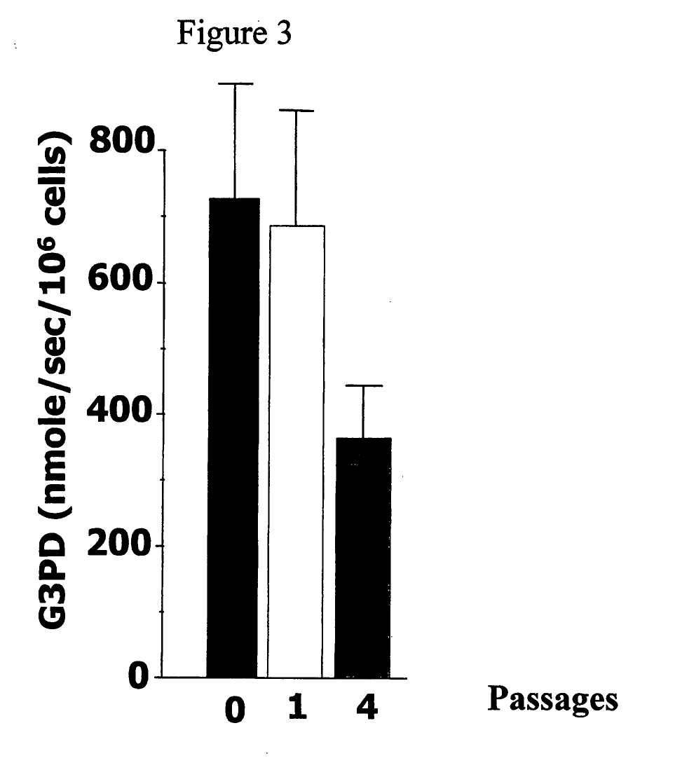

Next, glycerol-3-phosphate dehydrogenase (G3PD) activity was measured in cell homogenates from primary (0), first (1), and fourth (4) passage abdominal subcutaneous preadipocytes exposed to differentiation-inducing medium for 7 days. The data in FIG. 3 show an approximately 2-fold reduction in G3PD after only four passages. Thus, capacity for adipogenesis decreases with serial passage and, at later passage, preadipocytes undergo morphologic changes analogous to those of senescent fibroblasts.

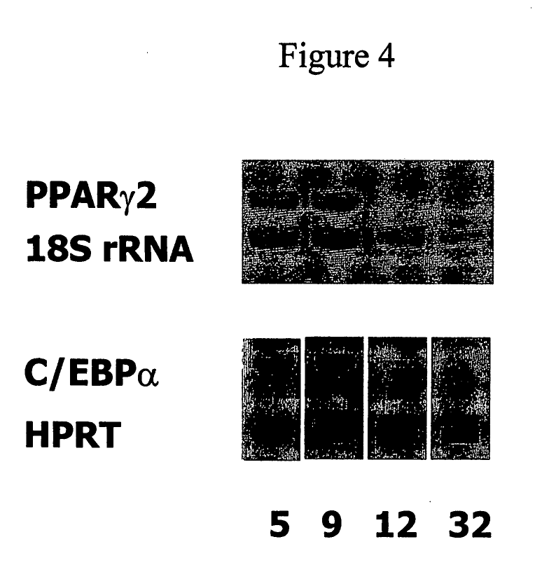

Finally, PPARγ2 and C/EBPα expression was assayed by competimer-based RT-PCR. The data in FIG. 4 demonstrate that PPARγ2 and C/EBPα mRNA levels decline in differentiating abdominal subcutaneous preadipocytes with increasing passage. 18S rRNA was measured as an internal control for PPARγ2, and hypoxanthine phosphoribosyl transferase (HPRT) mRNA as a control for C/EBPα.

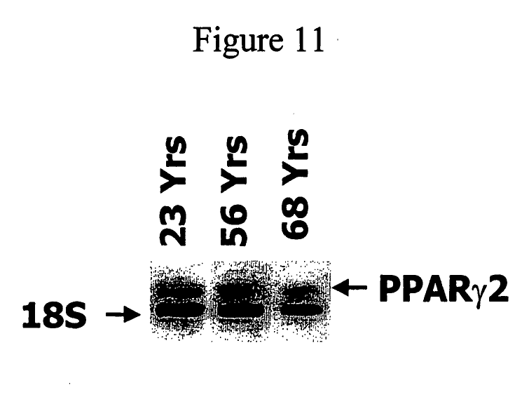

An analogous decline in PPARγ2 expression occurs with aging when differentiating primary preadipocytes cultured from subjects of various ages are compared. As can be seen in FIG. 11, PPARγ2 expression is lower in preadipocytes from older versus younger subjects.

### Example II

### Characterization of Telomere Reverse Transcriptase Activity and Expression in Primary Preadipocytes and Telomerase-Transfected Preadipocytes

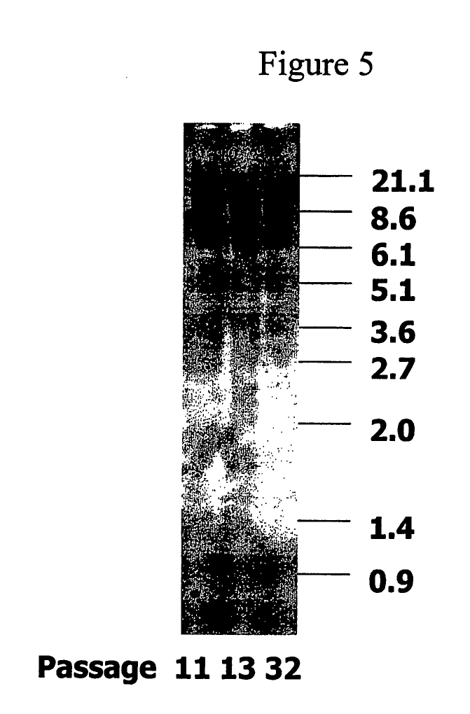

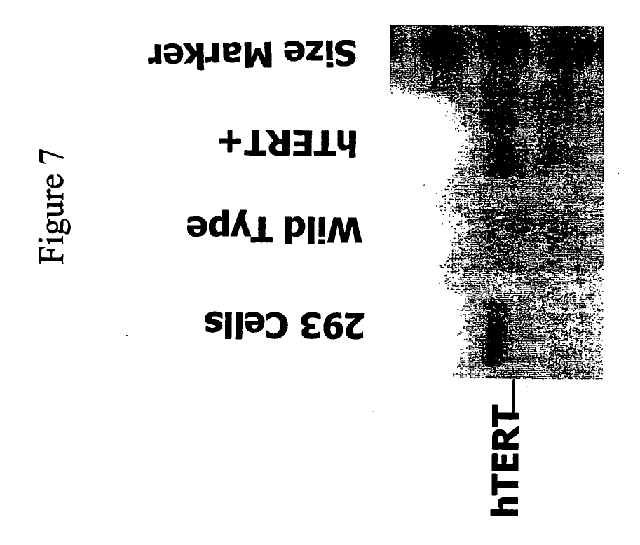

Initial studies were performed to determine telomere length and telomere reverse transcriptase (TERT) mRNA levels in serially passaged human preadipocytes. Southern blots of DNA from serially passaged preadipocytes showed that telomeric restriction fragment length shortens in wild type preadipocytes (FIG. 5). Preadipocyte telomerase shorten by approximately 120±44 bp per population doubling. Such decreases in telomere restriction fragment length have been reported in other cell types. These data indicate that primary preadipocytes do not contain significant human telomerase reverse transcriptase (hTERT) activity. Moreover, Northern blot analysis indicated that primary preadipocytes did not contain significant human telomerase reverse transcriptase (hTERT) mRNA after the 5th passage (FIG. 7, lane 2).

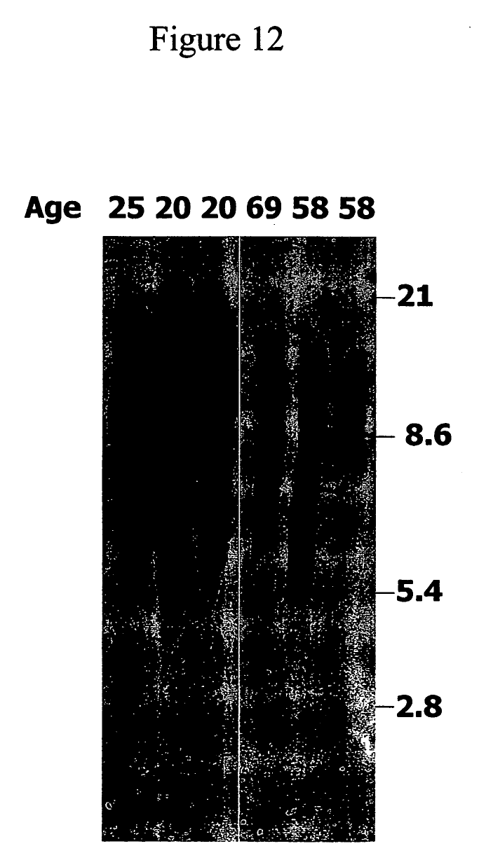

An analogous decline in telomere length occurs with aging when differentiating primary preadipocytes cultured from subjects of various ages are compared. As can be seen in FIG. 12, telomere length is shorter in preadipocytes from older than younger subjects. DNA was extracted from 4^{th} passage preadipocytes from 3 female subjects in their 20's and 3 older female subjects matched for BMI and ethnicity.

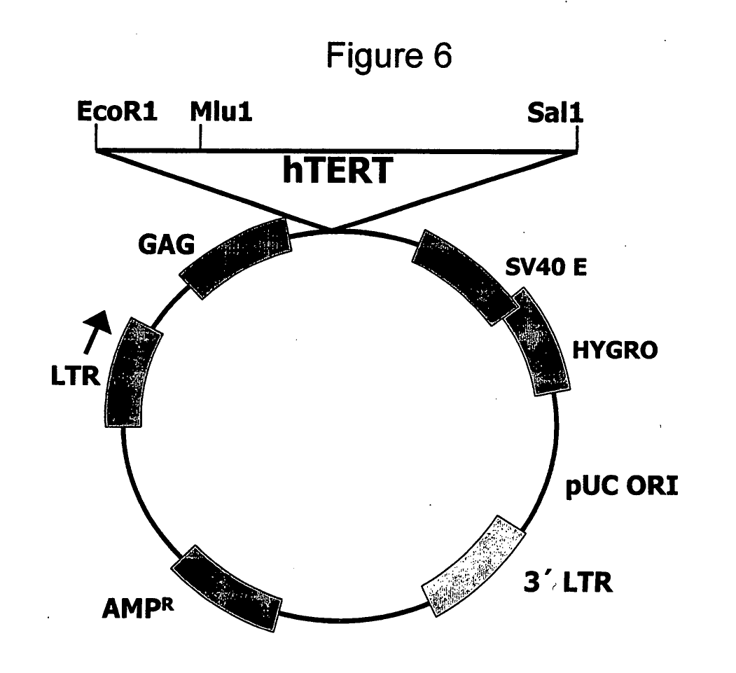

To test the hypothesis that telomere shortening contributes to declining capacities for adipogenesis and replication with serial passage, human preadipocytes were stably transfected with a telomerase-expressing construct. The construct comprised hTERT and hygromycin resistance sequences driven by the Moloney murine leukemia virus long terminal repeat and SV40 promoters, respectively. The construct is designated pBABE-hTERT-Hygro and is depicted in FIG. 6. Human abdominal subcutaneous preadipocytes that had undergone 7 population doublings were transfected and twenty two stably-transfected clones derived from single preadipocytes were prepared by selection for hygromycin resistance. Telomerase transfection was verified by detecting hTERT mRNA (FIG. 7). Briefly, expression of the human telomerase reverse transcriptase (hTERT) catalytic subunit-specific messenger RNA was measured by RT-PCR. The hTERT-transfected cells were serially passaged 10 times before telomerase mRNA was analyzed by RT-PCR (hTERT). Wild type human preadipocytes did not express detectable endogenous telomerase. RNA from cells of the immortal human kidney 293 line, which expresses a large amount of telomerase, was used as a positive control.

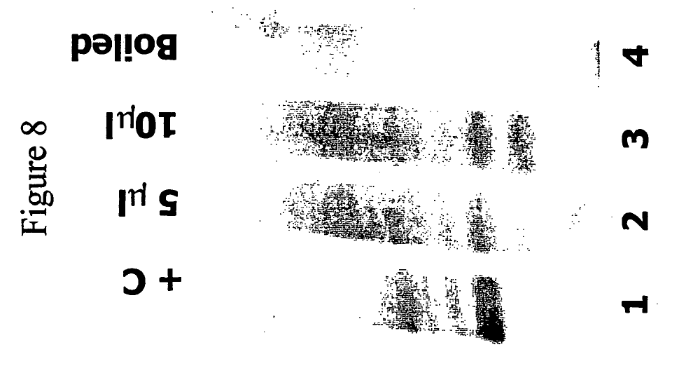

Telomerase transfection was also verified by detecting telomerase activity (FIG. 8). A terminal repeat assay protocol (TRAP) assay was performed to measure telomerase activity in an extract from hTERT-transfected cells. Telomerase activity increased with the amount of extract from transfected cells assayed (lanes 2 and 3). The extract contained 0.5 μg/μl protein. Activity disappeared following inactivation of telomerase in the extract by heat treatment (lane 4). As a positive control, an extract from immortal 293 cells was assayed (lane 1).

### Example III

### Capacity for Both Replication and Differentiation are Greatly Enhanced in Telomerase-Expressing Preadipocytes

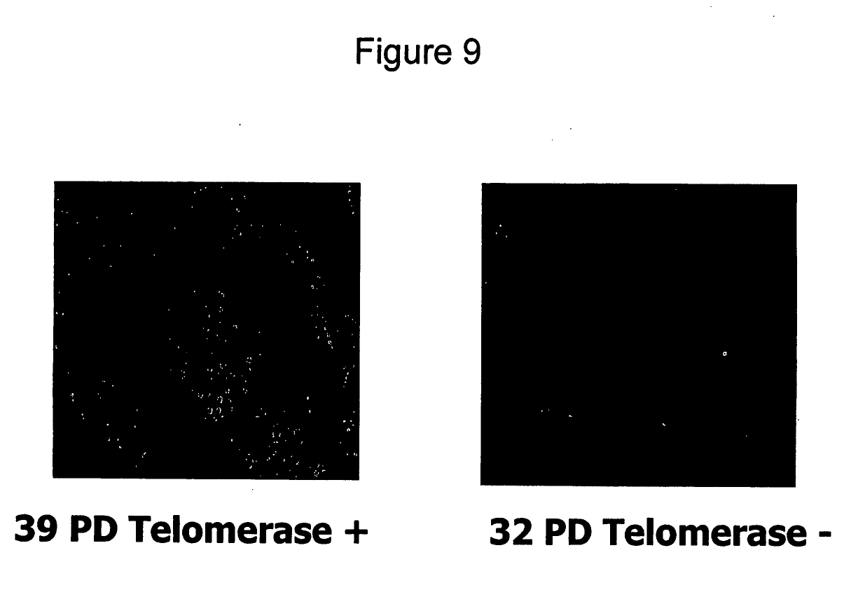

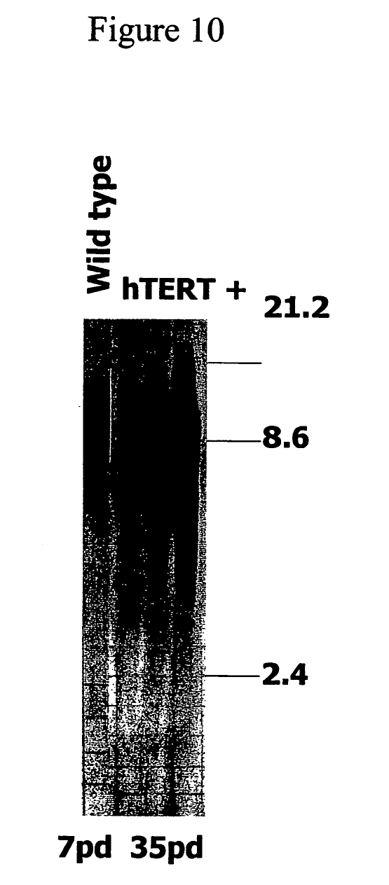

Telomerase-expressing clones (obtained from abdominal subcutaneous preadipocytes as described above) exhibited varying capacities for replication and differentiation. Telomerase-expressing clones were capable of being passaged 39 times over a two month period, while it took wild type cells two years to achieve 32 doublings. Some telomerase-expressing clones were capable of over 50 population doublings within 4 months before occurrence of replicative arrest. FIG. 9 demonstrates that clones that stably express telomerase retain capacity to differentiate for at least about 40 population doublings, while capacity for differentiation (i.e., capacity for adipogenesis) in wild type preadipocytes declines rapidly with serial passage. A high capacity for adipogenesis after 50 doublings, to an extent generally seen only in the first 5 doublings of primary culture preadipocytes, was found in clones capable of extensive replication. Differentiation of wild type cells exposed to differentiation media decreased in primary cultures of wild-type cells and was minimal by 6^{th} passage. Telomere fragment length was stable or increased after 35 population doublings (pd) in human abdominal subcutaneous preadipocyte clones ectopically expressing telomerase (hTERT+) compared to wild type 7th passage cells from the same subject (FIG. 10). Telomere restriction fragment length was assessed by Southern blotting. Thus, telomerase expression can forestall declining replicative potential and adipogenesis (e.g., capacity to acquire specialized function) in serially-passaged human preadipocytes, permitting generation of large numbers of differentiated cells from a single preadipocyte.

TABLE III
Telomerase Expression Enhances Preadipocyte
Replicative Potential

| | Untrans- | Telomerase | |
|---|---|---|---|
| | fected | Transfected | Significance |
| Population doublings at | 6 | 35 | |
| time of plating | | | |
| Cloning efficiency (%) | 19.6 ± 2.6 | 42 ± 0.9 | p < 0.00005 |
| Time between cloning and 1^{st} | 38 | 21 | |
| confluent colony (days) | | | |
| Cells resulting from each cell | 189 ± 46 | 1357 ± 210 | <0.01 |
| plated (means) | | | |
| Cells/colony (mean | 6.4 ± 2.7 | 16.0 ± 2.2 | p < 0.05 |
| cells × 10³) | | | |
| Cells achieving >14 | 15.3 ± 6.0 | 37.6 ± 6.0 | p < 0.05 |
| population doublings (% of | | | |
| clones with >16384 cells) | | | |

Table III demonstrates that telomerase expression enhances preadipocyte replicative potential. Telomerase expressing and untransfected abdominal subcutaneous preadipocytes from the same subject were cloned by plating 250 telomerase expressing cells in five 96 well plates and 300 untransfected cells in 6 plates. The telomerase-expressing cells had undergone 35 population doublings between the initial isolation from the subjects and cloning. The untransfected cells had only undergone 6 population doublings. The first colony arising from a telomerase expressing cell achieved confluence 21 days following plating, while the first untransfected clone to reach confluence took 38 days. Cloning efficiency was over 2 fold higher in the telomerase-expressing than untransfected cells. Cloning efficiency is expressed as the % of cells plated that had formed colonies within 21 days after plating. Despite their having been in culture for approximately half the time of the untransfected cells, the telomerase-expressing clones contained more than twice as many cells. Coupled with their higher plating efficiency, this resulted in the telomerase-expressing cells giving rise to over 7 times the number of daughter cells compared to untransfected preadipocytes. The proportion of telomerase-expressing cells that achieved 14 population doublings within 21 days was 2.5 fold higher than untransfected cells within 38 days. Thus, telomerase-expressing preadipocytes are capable of more rapid replication than wild type cells. Despite their more rapid replication, the maximum number of population doublings telomerase-expressing preadipocytes could achieve was limited: replicative arrest occurred by 55 population doublings. None of the 105 clones prepared from telomerase-expressing preadipocytes exhibited morphological features of transformation such as development of cell islands or lack of contact inhibition. This lack of transformation is in keeping with observations in other types of human telomerase transfected cells (Jiang et al. (1999) Nature Gen. 21:111-114).

### Example IV

### Telomerase Expression Prevents Declining Adipogenic Transcription Factor Expression with Serial Passage

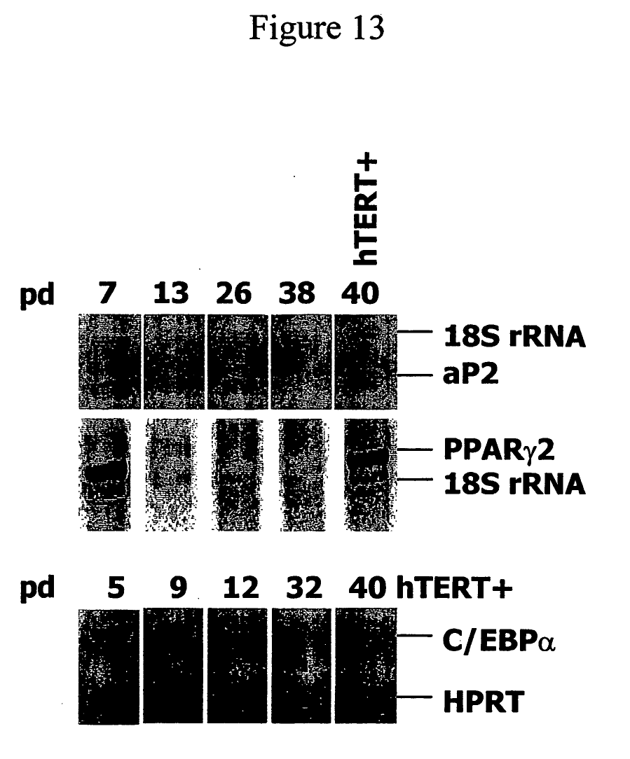

Telomerase expression prevented declining expression of aP2, a marker of differentiation whose expression is regulated by C/EBPα and PPARγ2, as preadipocytes were serially passaged (FIG. 13). Declining adipogenesis with serial passage was associated with decreasing expression of the adipogenic transcription factors, C/EBPα and PPARγ2. A decrease in C/EBPα and PPARγ2 mRNA levels was evident within 12 population doublings in wild type differentiating preadipocytes as shown in FIG. 13. However, expression of these adipogenic regulators was similar in 40^{th} passage telomerase expressing preadipocytes to that in 12^{th} passage wild type cells. Thus, enhanced expression of adipogenic transcription factors is one mechanism through which telomerase expression, resulting in telomere length maintenance, prevents decreasing adipogenesis.

Those skilled in the art will recognize, or be able to ascertain using no more than routine experimentation, many equivalents to the specific embodiments of the invention described herein. Where any concept(s) or element(s) of the invention is separately presented for convenience, it is understood that the combination of any such separately presented concept(s) or element(s), as necessary, is also encompassed by the invention. Such equivalents are intended to be encompassed by the claims.

The contents of the patents and references cited throughout this specification are hereby incorporated by reference in their entireties.

1. A primary preadipocyte strain, wherein said strain expresses telomerase reverse transcriptase (TERT) such that said strain maintains replicative potential and adipogenic capacity.

2. The preadipocyte strain of claim 1, wherein said strain is a human preadipocyte strain.

3. The preadipocyte strain of claim 1, wherein the TERT is human telomerase reverse transcriptase (hTERT).

4. The preadipocyte strain of claim 1, wherein said strain maintains replicative potential and adipogenic capacity over at least 40 population doublings.

5. The preadipocyte strain of claim 1, wherein said strain maintains replicative potential and adipogenic capacity over at least 50 population doublings.

6. A primary preadipocyte strain, wherein said strain expresses telomerase reverse transcription (TERT) such that said strain has enhanced replicative potential and maintains adipogenic capacity.

7. The preadipocyte strain of claim 6, wherein said strain is a human preadipocyte strain.

8. The preadipocyte strain of claim 6, wherein the TERT is human telomerase reverse transcriptase (hTERT).

9. The preadipocyte strain of claim 6, wherein said enhanced replicative potential is measured as the ability to achieve at least 40 population doublings in less than one year.

10. The preadipocyte strain of claim 6, wherein said enhanced replicative potential is measured as the ability to achieve at least 50 population doublings in less than one year.

11. The preadipocyte strain of claim 6, wherein said enhanced replicative potential is measured as the ability to achieve at least 40 population doublings in less than six months.

12. The preadipocyte strain of claim 6, wherein said enhanced replicative potential is measured as the ability to achieve at least 50 population doublings in less than six months.

13. The preadipocyte strain of claim 6, wherein the strain has an enhanced adipogenic potential, wherein the enhanced adipogenic potential is measured by the ability to maintain expression of adipogenic transcription markers for at least 40 passages.

14. The preadipocyte strain of claim 6, wherein the strain maintains expression of TERT for at least 40 passages.

15. A method for producing a primary preadipocyte strain which maintains replicative potential and adipogenic capacity, comprising engineering primary adipocytes to express telomerase reverse transcriptase (TERT) such that said strain maintains replicative potential and adipogenic capacity.

16. A method for producing a primary preadipocyte strain which maintains replicative potential and adipogenic capacity, comprising engineering primary adipocytes to express telomerase reverse transcriptase (TERT) such that said strain maintains replicative potential and adipogenic capacity over at least 40 population doublings.

17. The method of claim 16, wherein said strain maintains replicative potential and adipogenic capacity over at least 50 population doublings.

18. A method for producing a primary preadipocyte strain having enhanced replicative potential and maintained adipogenic capacity, comprising engineering primary adipocytes to express telomerase reverse transcriptase (TERT) such that said strain has enhanced replicative potential and maintains adipogenic capacity.

19. The method of claim 18, wherein said enhanced replicative potential is measured as the ability to achieve at least 40 population doublings in less than one year.

20. The method of claim 18, wherein said enhanced replicative potential is measured as the ability to achieve at least 50 population doublings in less than one year.

21. The method of claim 18, wherein said enhanced replicative potential is measured as the ability to achieve at least 40 population doublings in less than six months.

22. The method of claim 18, wherein said enhanced replicative potential is measured as the ability to achieve at least 50 population doublings in less than six months.

23. A method for engineering primary adipocytes to maintain replicative potential and adipogenic capacity, comprising:
a) introducing into said adipocytes a nucleic acid that encodes telomerase reverse transcriptase (TERT); and
b) selecting for adipocytes that express TERT.

24. A preadipocyte strain obtained by a process comprising:
a) introducing into an isolated primary preadipocyte population a nucleic acid that encodes telomerase reverse transcriptase (TERT);
b) selecting from said population a preadipocyte that expresses TERT; and
c) replicating said preadipocyte that expresses TERT.

25. The method of any one of the preceding claims, wherein said primary adipocytes are human primary adipocytes.

26. The method of any one of the preceding claims, wherein said TERT is human telomerase reverse transcriptase (hTERT).

27. A method to identify adipogenic modulators comprising, contacting the strain of any one of the preceding claims with a test compound and determining an effect on said strain, such that a modulator is identified.

28. A method to identify adipogenic modulators comprising, contacting a cell from the strain of any one of the preceding claims with a test compound and determining an effect on said cell, such that a modulator is identified.

29. A cell from a strain or a strain according to any one of the preceding claims, for use in cosmetic or reconstructive surgery.

30. A cell or strain according to any one of the preceding claims, for use in administering a therapeutic agent.

31. The cell or strain of claim 30, wherein the therapeutic agent is a secreted protein.

32. The cell or strain of claim 31, wherein amount of the secreted protein is enhanced as compared to a suitable control.

33. The cell or strain of claim 30, wherein the therapeutic agent is selected from the group consisting of: hormones, growth factors, cytokines, enzymes, cholesterol binding proteins, cholesterol removing proteins, and combinations thereof.

34. The cell or strain of claim 30, wherein the therapeutic agent is an adipocytokine.

35. The cell or strain of claim 34, wherein the therapeutic agent is adiponectin.

36. The cell or strain of claim 30, wherein the therapeutic agent is insulin.

37. A primary preadipocyte which is co-transfected with telomerase reverse transcriptase (TERT) and a secreted polypeptide-encoding nucleic acid.

38. A method of delivering a polypeptide to a mammal comprising administering to said mammal the preadipocyte of claim 37.

39. A preadipocyte strain for delivery of a polypeptide to a mammal, wherein said strain has been engineered to express TERT and said polypeptide.

40. The strain of claim 39, wherein said strain has been engineered to express a secreted polypeptide.

41. A method of delivering a polypeptide to a mammal comprising administering to said mammal the strain of claim 39 or 40.

42. The method of claim 41, wherein the strain is differentiated prior to administering the strain.
